(19) United States
(12) Patent Application Publication
(10) Pub. No.: US 2003/0212359 A1
(43) Pub. Date: Nov. 13, 2003

(54) Conformable bi-laminate compression bolster and method for using same

(76) Inventor: Charles E. Butler, Houston, TX (US)

Correspondence Address: Michael J. Smith, VINSON & ELKINS LLP, 2300 First City Tower, 1001 Fannin, Houston, TX, 77002-6760, US

(21) Appl. No.: 10/385,197

(22) Filed: Mar. 7, 2003

Related U.S. Application Data
(60) Provisional application No. 60/362,350, filed on Mar. 7, 2002.

(51) Int. Cl. A61F 13/00; A61F 15/00

(52) U.S. Cl. 602/48

(57) ABSTRACT

A bolster dressing comprising an inner conformable layer and an outer semirigid layer for use in closing wounds and securing skin grafts or surgical flaps. This bolster dressing reduces edema, hematoma, seroma and shear of the wound bed.

### CROSS-REFERENCE TO RELATED APPLICATIONS

The present application is a continuation of provisional U.S. Ser. No. 60/362,350 filed Mar. 7, 2002.

### FIELD OF THE INVENTION

The present invention relates to the field of wound healing, specifically surgical wound closure. More particularly, the invention relates to bolster dressings for closing and securing skin grafts, surgical flaps and other types of wounds.

### STATEMENT REGARDING FEDERALLY SPONSORED RESEARCH OR DEVELOPMENT

N/A

### BACKGROUND OF THE INVENTION

Skin grafting is the most common procedure used to reconstruct wounds that are too large to close primarily. These skin grafts must be anchored and compressed to the underlying wound bed for 4-14 days after application during the healing process. The first several days are most crucial for the graft to 1) initially adhere, 2) maintain its viability by receiving nutrients from the wound fluid, 3) develop its own blood supply from the wound bed and 4) attach and heal permanently to the wound. Typically, skin grafts are secured using a bolster dressing. This provides mild compression to facilitate adherence and prevent bleeding and fluid collections, and immobilization to prevent shear or separation. Traditional bolster dressings can be difficult to apply to large, uneven, convex or irregular surfaces. In addition, traditional dressings such as cotton or gauze contain little or no surface rigidity and are typically applied by tie-over sutures placed at the perimeter of the graft, causing a round, or concave shaped bolster surface over the graft. This configuration places perpendicular compressive force only at the central point or axis and not at the peripheral areas of the graft. This method does not allow skin grafts to be secured to the graft bed and maintain a flat planar surface, particularly as graft size increases, nor are such dressings easily applied to concave, convex or other contoured areas.

Neither have semi-rigid materials proven effective for skin graft bolsters. These materials are generally not conformable enough to an irregular surface to be used successfully. In addition, methods of securing the bolster often create focal areas of intense compression leading to focal necrosis and skin graft death.

The difficulty in performing a successful skin graft and having it survive and heal lies not only in the irregularity of the surfaces that are to be treated, but also in the movement and muscle contraction to which the graft is likely to be subjected. The difficulties with movement are best illustrated by considering the human tongue, which is constantly moving with both extrinsic and intrinsic muscle contractions in essentially unlimited orientations, resulting in an extensive variation in length, width and thickness. This constant dynamic alteration in surface dimensions creates difficulties in anchoring and immobilization of skin grafts to body surface defects.

Various methods have been used in the attempt to immobilize a split thickness skin graft on the tongue. Staples, rubber bands, tape, glue and safety pins have all been used (Rees, Plast. Reconstru. Surg. 43:635, 1969; Freeman, Plast. Reconstruct. Surg. 30:289, 1962, Bromberg, Surgery, 55:846, 1964). In one method, a piece of gauze may be stapled to the graft around the edges and then sutures laced through the staples to secure the gauze to the graft. Another method of immobilizing split skin grafts to the tongue that has been described is multiple anchoring sutures of cat-gut through the graft in a pattern that creates the overall appearance of a quilted graft. With split-thickness skin grafts (STSG) of the tongue, for example, resorbable quilting sutures can provide multiple fixation points to limit the potential for separation and shear. Traditional tie-over bolsters are also used to immobilize intraoral STSGs but are difficult to use on large surface area grafts or those which extend over curved or complex surfaces.

None of these methods, however, provide a uniform pressure perpendicular to the graft, to hold the entire graft area against the wound bed, nor do they immobilize the wound area to reduce shear, hematoma, seroma or wound separation.

### SUMMARY

An aspect of the present disclosure is a bolster dressing used to secure skin grafts to wounds (graft beds), or to protect wounds, particularly surgical wounds, and further to immobilize or restrict movement in areas of wounds or grafts. The disclosed devices may also be used as compression devices to reduce or inhibit lymphedema and edema. The preferred bolster includes at least two laminated components. A first component is one or more external semi-rigid or rigid members that maintain a flat surface even when compressed, to direct compressive forces evenly and perpendicularly to their entire surface plane. The external members preferably exhibit enough flexibility or malleability to conform to gentle curvatures in the overall contour of the wound or skin graft bed. The bolster is designed so that the force on the graft can be directed perpendicular to the tangent of these curved regions, thus compressing the graft onto the graft bed at right angles to its surface. This configuration provides uniform, direct perpendicular forces and prevents shearing between the skin graft and the graft bed and reduces hematoma, seroma and wound separation.

A second component of the preferred bolster is one or more inner members that are composed of a soft, conformable material such as a conformable polyurethane foam. The inner member creates a compression buffer protection system to prevent focal areas of increased pressure, which can cause graft necrosis (death). The inner layer also molds exactly to the surface irregularities and undulations further distributing the compressive force evenly and in the appropriate perpendicular orientation. The even pressure provided by the bolster reduces shear, separation and bleeding between the skin graft and graft bed. Materials that may be used for the inner layer include, but are not limited to soft foam, cotton, petroleum jelly impregnated gauze, and combinations of these.

The bi-laminate, graduated compression bolster of the present disclosure can be constructed quickly, easily and inexpensively in multiple configurations. The shape, size, thickness, and relative rigidity can be altered to address various specific application areas. In addition, the semi-rigid component can be pre-contoured with the appropriate curvature to fit specific body parts or areas. Examples of specific areas of the body where skin grafting is difficult due to contour irregularity and/or motion, and in which the use of the preferred bolsters of the present disclosure would be particularly beneficial include, but are not limited to the groin, perineal or vulvar area, the hand, foot, fingers, toes, and webspaces between the thumb and hand, any large and/or flat surfaces such as the thigh or chest wall, the ear, tongue or cheek area, the nipple or areola, the axilla (armpit) or cubitis (elbow) areas.

The bolsters of the present disclosure offer the further advantage of providing stenting or splinting properties due to the semi-rigid or rigid outer member, as the rigidity of the attached bolster can limit local movement of tissue to reduce shear forces applied to the skin grafted area. The bolster may be applied and secured using multiple techniques known in the art including tie-over sutures, trans-tissue bolster sutures, staples, adhesive film, wrapping, tape, casts or splints, silicone gel or elastic materials and can be applied both above and below the grafted tissue in certain areas such as the tongue or ear, for example. The bolster may also have negative pressure applied to one layer using a vacuum assisted closure device, preferably applying negative pressure via a catheter. The two sided bolster can be constructed as a single device that surrounds or encloses the tissue graft, or it can be two devices that are applied on opposing sides of the tissue graft using trans-bolster sutures, for example. The application of a bolster to both sides of a graft area is particularly useful in areas of high mobility such as the tongue and near mobile joints such as the finger and toe areas.

Another advantage offered by the disclosed bolsters is that the bolsters compress and splay tissue out to its maximum length and width, which can reduce subsequent graft contraction and reduce the magnitude of surface contraction. Immobilization of tissue with the disclosed devices reduces the shear, separation and bleeding between the skin graft and graft bed, thus maximizing the likelihood of graft survival and success. Highly irregular surface contours are compressed by the intervening soft compressible inner layer to prevent focal “over compression,” which leads to graft or tissue necrosis, or “under compression,” which leads to skin graft separation from graft bed, hematoma or seroma. Blood clots or fluid collection under the graft lead to skin graft death.

It is an aspect of the present disclosure that specific products may be manufactured to suit specific needs or applications. For example, pre-cut bolsters may be supplied to match particular areas of the body such as ears, tongue, cheek, nipple areola, fingers, toes, groin or vagina. Alternatively, the material may be supplied in large sheets to be shaped by the physician to fit a particular need. The semi-rigid components may also be provided with pre-shaped contours to fit particular curved surfaces such as the fingers or tongue. A particular bolster is also manufactured for use in nipple/areola reconstruction or any reconstruction of the breast, such as breast reconstruction after mastectomy, breast reduction or lift.

The described bolsters may also be used for protection and healing of wounds, including surgical wounds, and/or surgical flaps. A surgical flap is generally known in the art as any vascularized tissue including skin, fat, muscle, bone, or other tissue that has been moved from one location of the body to another location. The disclosed devices provide compression, immobilization, protection from mechanical injury and the ability to deliver active agents to any such wound or surgical site. The devices further provide protection from hematomas, seromas and shear, thus aiding the healing process.

In a preferred embodiment the disclosure provides a bolster designed for use in nipple/areola reconstruction. Commonly the nipple is reconstructed with adjacent tissue flaps yielding a fragile and delicate structure, prone to trauma. A circular skin graft is often used surrounding the nipple reconstruction to form the areola. A circular bi-laminate bolster as disclosed herein with an umbilicated or hollow center is used to provide evenly distributed compression perpendicular to the areola skin graft as well as surround and protect the delicate nipple reconstruction. The bolster is preferably a round, donut shaped product with a hole in the center to fit over the nipple. The device is preferably taped to the skin to apply pressure and to hold it in place. In addition, an adhesive such as a silicone material either integrated with or supplied separately from the inner foam layer may be applied to the soft foam layer to increase adhesion to the skin. Such silicone materials have also been shown to reduce the occurrence of and assist in the treatment of scarring commonly associated with surgical wounds or skin grafts. In alternative embodiments, a device for use in protecting the nipple area after surgery may resemble a cone or egg that fits over the breast. In this embodiment the outer shell is constructed of a rigid or semi-rigid material and encloses a soft foam that approximates the shape of the breast. During use the breast is placed in the device and the soft foam inner layer conforms to the shape of the breast and a void is provided for the nipple. After attachment, the device provides compression for grafted skin and protects the breast area from trauma, shear, rubbing by clothes, etc. or accidental bumping. The outer semi-rigid layer may also be of greater or lesser diameter than the inner conformable layer and the inner conformable layer may also be tapered from the area nearest the nipple to the outer edge of the bolster. Where the outer semi-rigid layer is larger in diameter than the inner conformable layer, then the outer semi-rigid layer may contain an adhesive around the outer rim to attach the bolster to the skin.

An example of a bolster used in a skin graft on a tongue is described in more detail below.

Any suitable material may be used in the construction of the disclosed devices. The preferred material for the inner layer is a soft malleable foam such as a polyurethane foam. Exemplary soft polyurethane foam products are currently marketed by 3M Health Care under the trade name Reston® and by Mölnlycke Health Care under the trade name Mepilex®. A preferred soft foam is a semi-open celled foam that is deformable to conform to irregularly shaped tissue or body areas, is compressible, and is durable enough to remain in place for up to seven days. Typical foams are those that deform under pressure and slowly return to their original shape when the pressure is removed. Other products that may be used include cotton, wool, woven or unwoven synthetic materials, silicone gel or a vacuum assisted closure dressing suited for use with a vacuum assisted closure device. An exemplary vacuum assisted closure device is currently marketed by Kinetic Concepts, Inc. under the trade name V.A.C.® Therapy. The inner layer materials may be absorbent effective to remove blood and other fluids produced by the wound, or they may be non-absorbent, or hydrophobic, when used in areas such as the oral cavity in which excess moisture exists and might be wicked to the wound by an absorbent material. Blood and other fluid produced by the wound may also be effectively removed where a vacuum assisted closure device is employed as the negative pressure will pull the fluids away from the wound or skin graft region thus reducing edema, seroma or hematoma. The material of the inner layer is preferably sterile, non-allergenic, and non-toxic.

The semi-rigid or rigid outer layer may also be of any suitable material. Preferred materials include, but are not limited to semi-rigid polyethylene foams, polymers, plastics, woods, and metals such as aluminum or steel. An exemplary semi-rigid material is the low-temperature thermoplastic, Aquaplast (WFR/Aquaplast Corp.). The preferred material may be bent or shaped to the appropriate shape and is then rigid enough to hold that shape during the use of the bolster. The inner and outer layers may be bonded by an adhesive or by an epoxy type glue or they may be joined by a heat meld, for example. The device may be provided in a bonded state or the two layers may be supplied separately and optionally with an adhesive pre-applied to one or both layers.

In certain embodiments, the disclosed devices include a non-adhesive layer that separates the soft inner layer from the skin graft. The non-adherent layer may be a non-adherent gauze or a non-adherent film as are well known in the art. Exemplary non-adherent layers are silicone sheetings including those currently marketed by Mölnlycke Health Care under the trade names Mepilex®, Mepiform®, and Mepitel®, those currently marketed by Kendall under the trade name Xeroform™, those currently marketed by Johnson & Johnson Medical under the trade name ADAPTIC™, and those currently marketed by Winfield Laboratories under the trade name N-TERFACE®.

Certain devices may also include a drug delivery layer. A drug delivery layer may be disposed between the inner and outer layers, between the inner layer and the skin graft, or between the inner layer and a non-adherent layer. The drug delivery layer, if present, may be made of any suitable material, preferably a bio-compatible material, and in certain embodiments, of a biodegradable or bio-absorbable material. The drug delivery layer may comprise a polymer such as a solvent based polymer or a water based polymer and may include active agents within a porous structure, or enclosed or entrapped in or associated with microcapsules, microspheres, membranes, a gel, hydrogel, liposomes, cellulose, polymers such as glycosaminoglycans or other carbohydrate or protein containing carriers or capsules. The active agents may include, but are not limited to, drugs, hormones, antibiotics, growth factors, cytokines, chemotherapeutic agents, radiotherapeutic agents, antiseptics, anesthetics, vitamins, minerals, cellular nutrients, co-factors, antioxidants, deodorants, neutralizing agents, and combinations of these. In certain preferred embodiments the drug delivery layer is constructed to release the active agents over a period of hours or days, or in certain embodiments the drug delivery layer may be connected to a tube or other drug delivery device to replenish the agent supplied to the device on a periodic or continuous basis. In certain embodiments, a catheter or tube used as part of a vacuum assisted closure device may be used to replenish agents supplied to the device. It is also understood that the drug delivery layer and the inner layer of soft material may be separate laminated layers or they may be one and the same layer.

In certain embodiments, the disclosed devices include a vacuum assisted closure dressing suitable for use with a vacuum assisted closure device. This vacuum assisted closure dressing may be the inner conformable layer, the drug delivery layer, the non-adhesive layer, or a distinct and separate layer of the device. The vacuum assisted closure dressing may be adsorbent or nonadsorbent. The vacuum assisted closure dressing may further be used in conjunction with a vacuum-assisted closure device capable of applying negative pressure to the vacuum assisted closure dressing, preferably through the use of a catheter. The negative pressure provided by a vacuum assisted closure device provides a gentle even compression to the skin graft or wound bed thus reducing edema, hematoma, seroma, and shear or trauma of the tissue.

The devices may be applied or attached to the body by any means known in the art, including but not limited to sutures, staples, adhesive film, wrapping, tape, an elastic material, or a vacuum assisted closure device. In certain embodiments the bolsters are applied to both sides of skin graft or wound and may be attached by through and through sutures.

As used herein, the terms “skin” or “skin graft” are meant to refer to a full thickness or split thickness skin graft, and also to artificial skin or skin substitutes, including dermal and/or epidermal substitutes. Such materials may include cultured epithelial autografts, or composite grafts comprising keratinocytes, fibroblasts, and other cells, and may include collagen based membranes, allodermis, de-cellularized allodermis, xenogenic skin products, or other cutaneous skin products. Such materials include AlloDerm™ (acellular human cadveric dermis, LifeCell Corporation), Surgisis™ (Cook Group, Inc.), Dermagraft® (Smith & Nephew), Apligraf® (Novartis and Organogenesis), Epicel™ (Genzyme), and Orcel™ (Ortec International, Inc.).

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is an exploded perspective view of a preferred embodiment of the invention used in a tongue skin graft.

FIG. 2 is a schematic drawing of a prior art method of protecting a tongue skin graft in which focal pressure leads to ischemia.

FIG. 3 is a schematic drawing of a preferred embodiment of the invention for use on a tongue.

FIG. 4 is a schematic drawing of a prior art method of covering a skin graft in which cross sutures provide uneven pressure on the bolster.

FIG. 5 is a schematic drawing of a preferred embodiment of the invention.

FIG. 6 is a schematic drawing of a preferred embodiment of the invention designed for use in the ear.

FIG. 7 is a schematic drawing of preferred embodiments of the invention designed for use in the groin and perineum areas of the body.

FIG. 8 is a schematic drawing of a preferred embodiment of the invention designed for use in the axilla.

FIG. 9 is a schematic drawing of preferred embodiments of the invention designed for use in the dorsum, digit and webspace areas of the hand. Analogous bolsters may be used in the foot and toes.

FIG. 10 is a schematic drawing of preferred embodiments of the invention designed for use in the breast and nipple areas. The embodiment on the left is a donut shaped bolster and the embodiment on the right is a cone shaped bolster.

### DETAILED DESCRIPTION

The present disclosure includes a description of novel bolsters and dressing to be used in the treatment of wounds, surgical flaps, and skin grafts, including grafts of skin, skin substitutes, artificial skin and allodermis or other wound healing products. The dressings may also be used to cover and protect other primary wounds, including surgical wounds. Described are laminated layers of materials, including an outer layer of rigid or semi-rigid material and an inner layer of soft conformable material that is applied directly to the wound or skin, or that is separated from the skin or wound by a non-adherent layer, an active agent delivery layer or both.

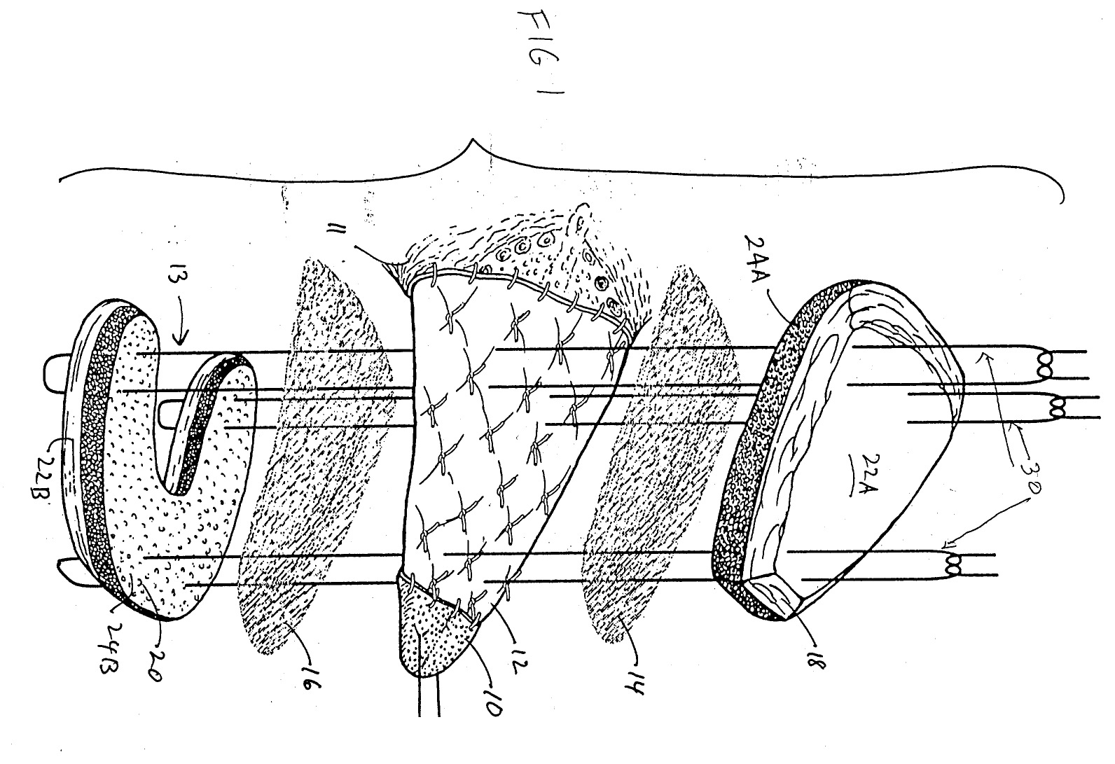

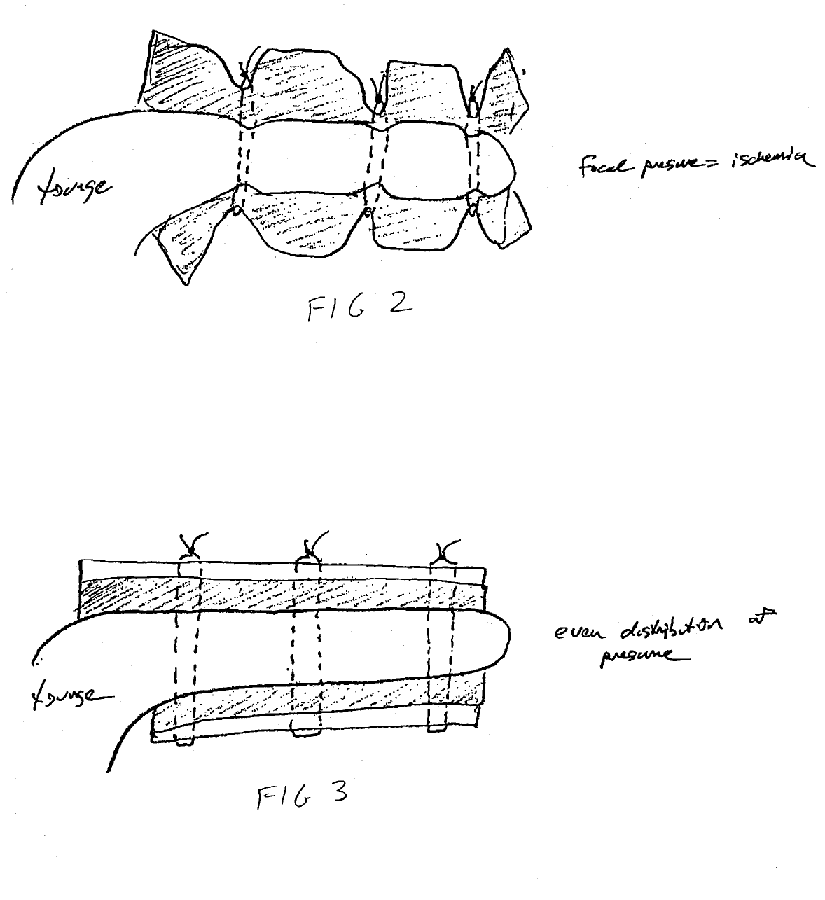

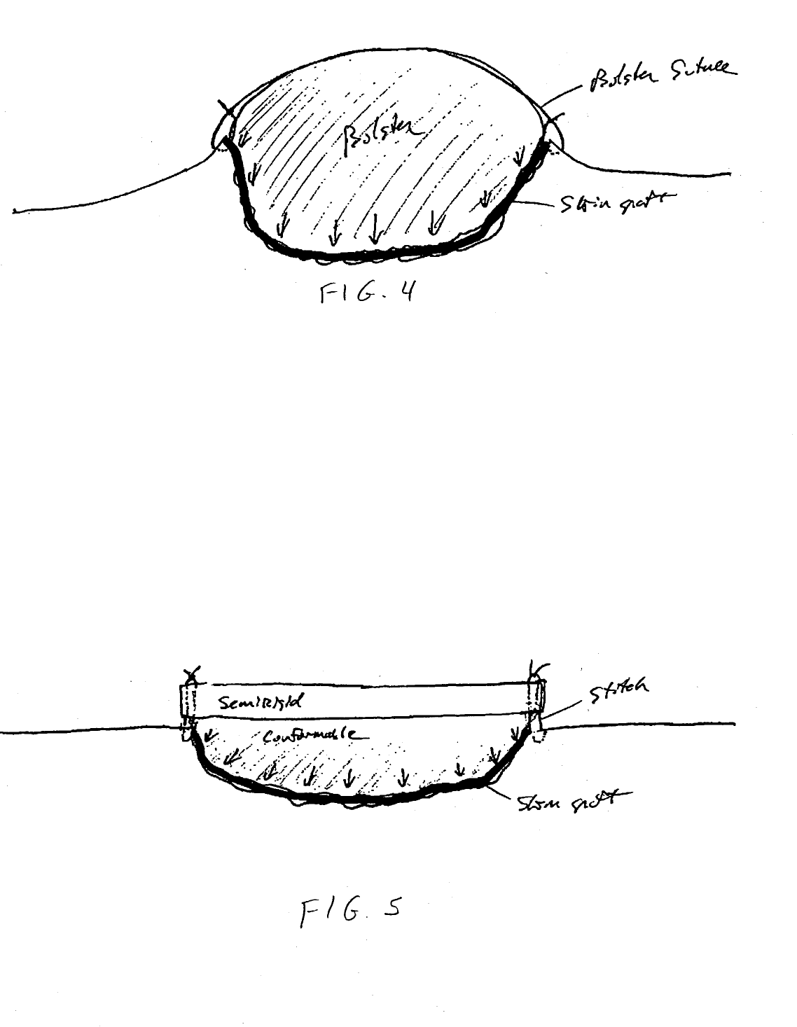

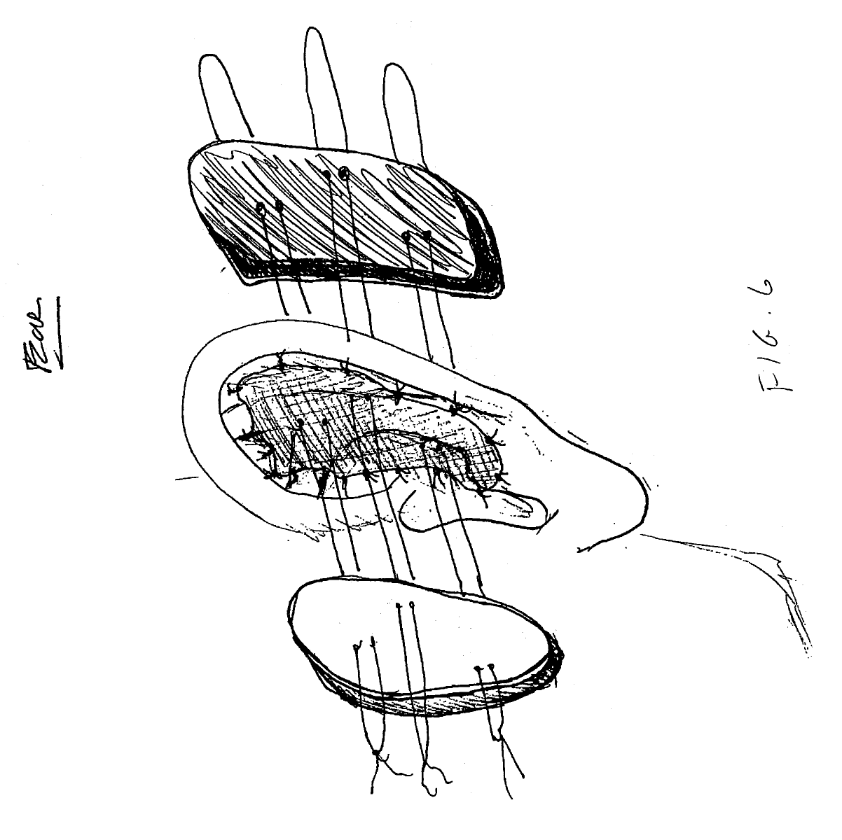

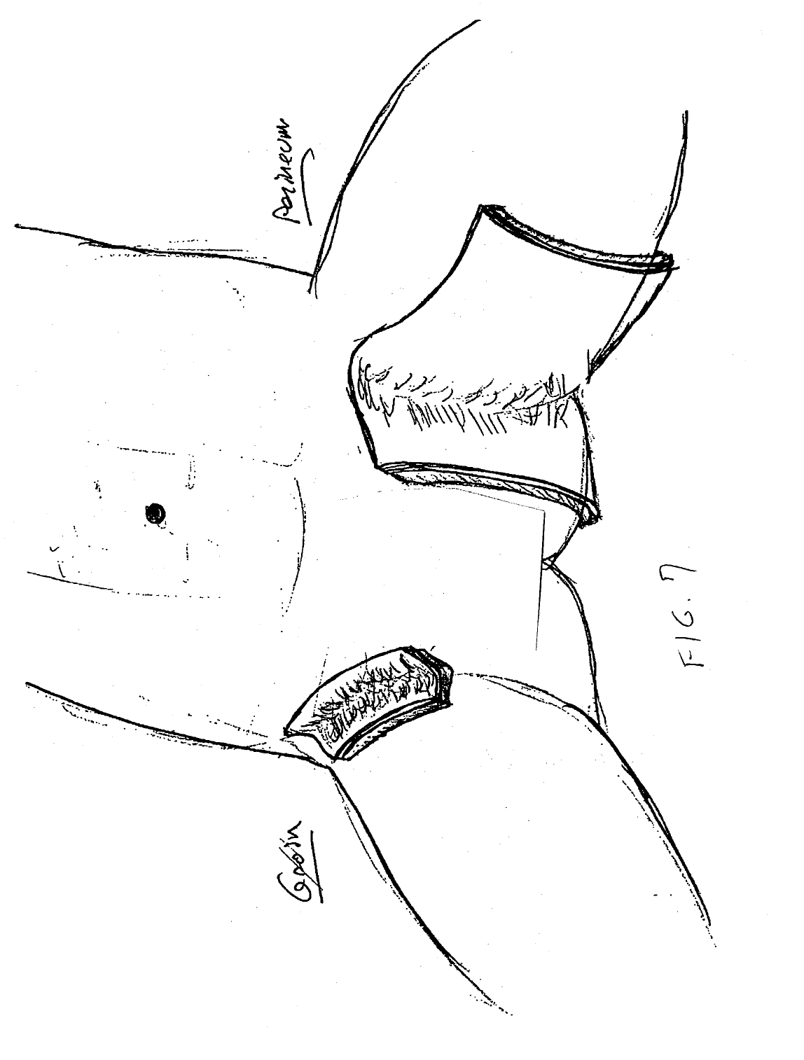

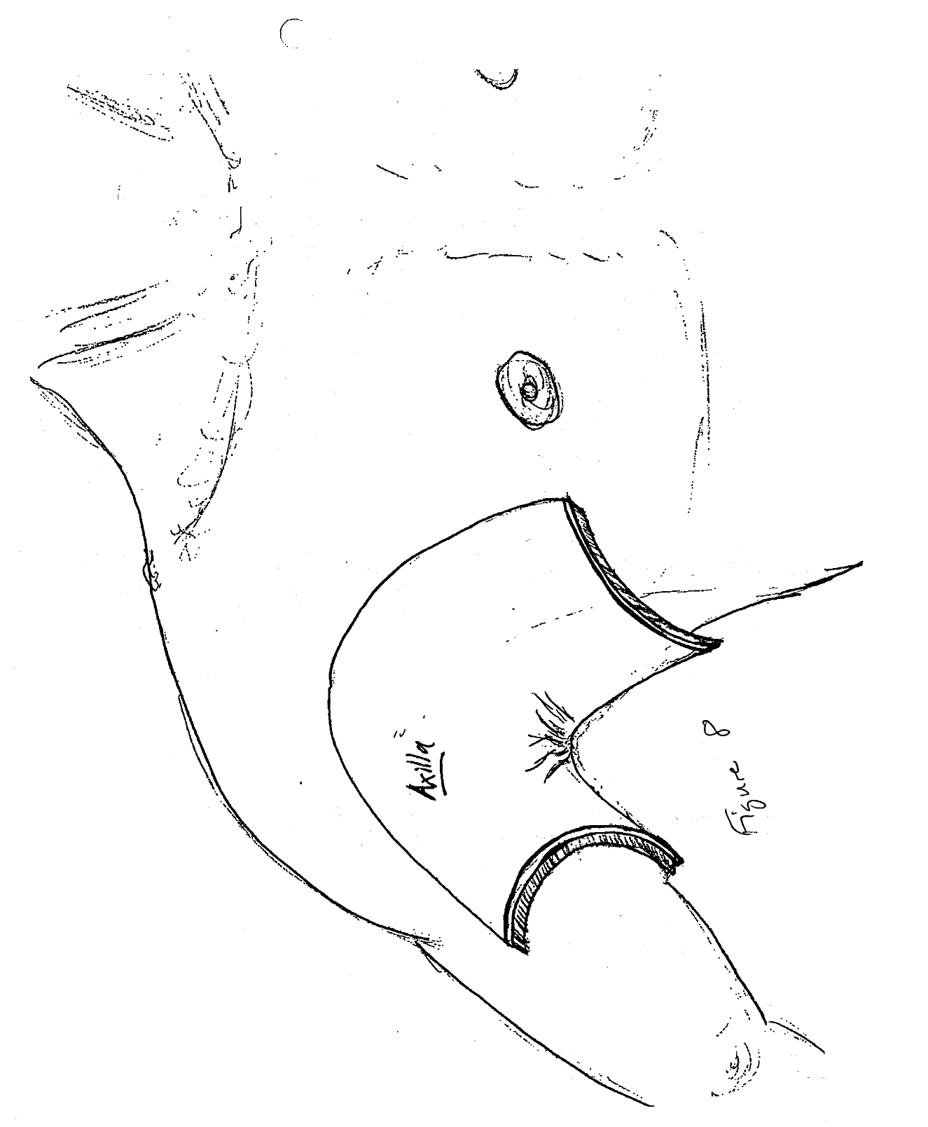

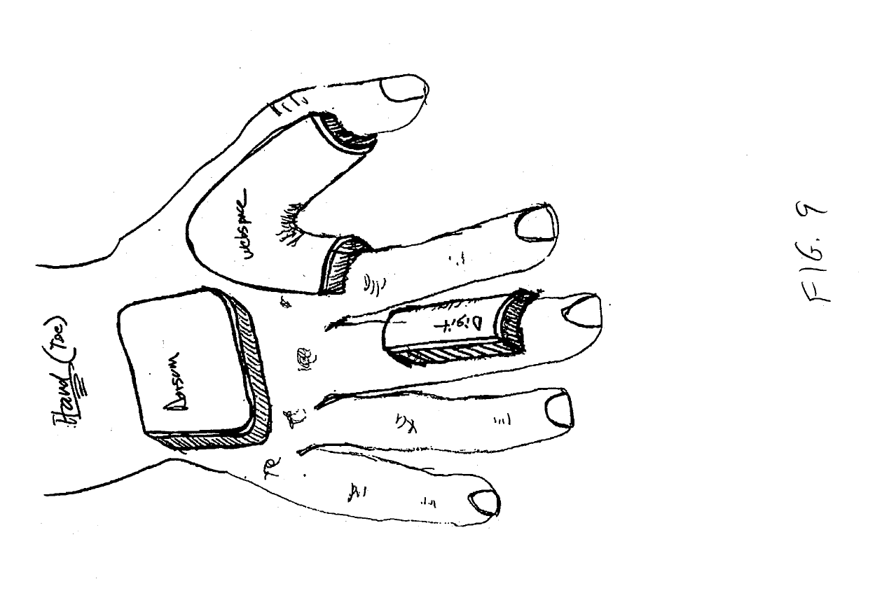

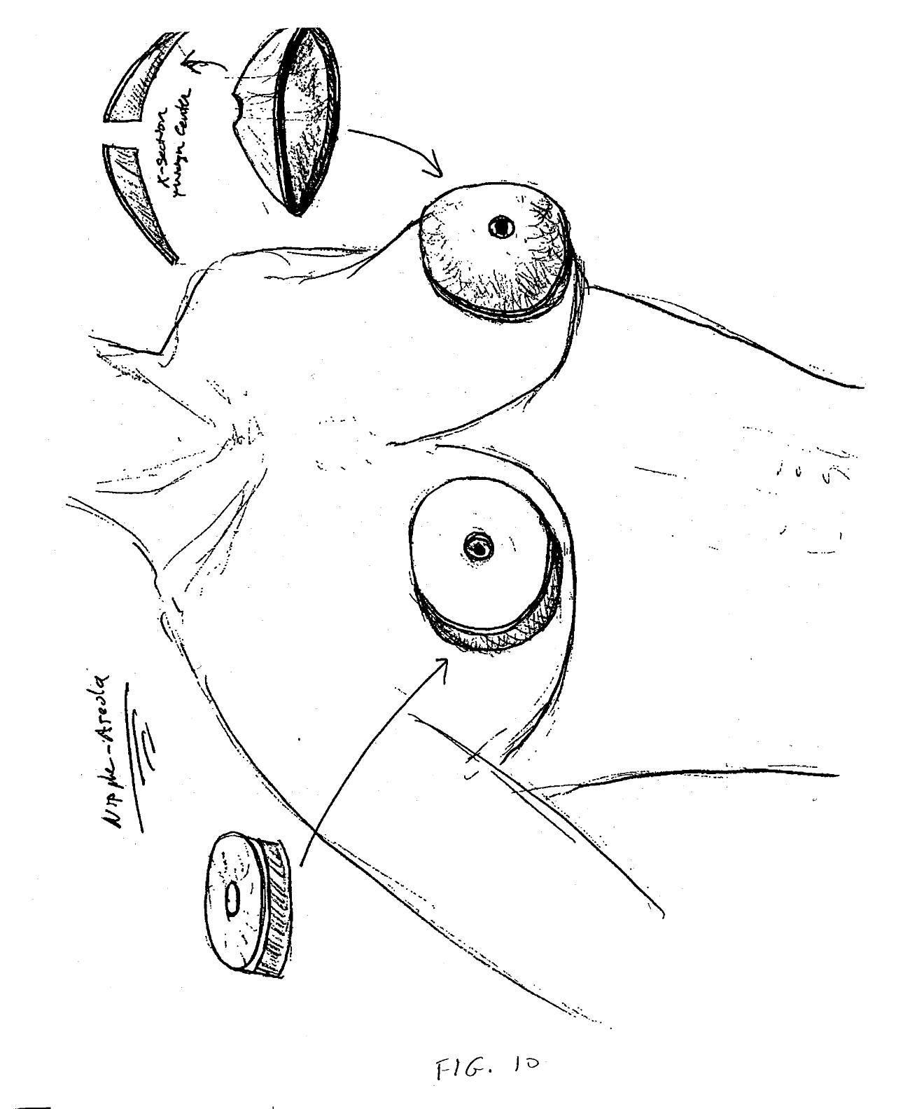

Shown in FIGS. 1-10 are embodiments of the invention configured for applications to various areas of the human body. It is understood, of course, that the invention is equally applicable to veterinary medicine and the treatment of animals in both clinical and experimental settings. Although the devices may be shaped and contoured from a large sheet material at the time of use, it is also an aspect of the disclosure that kits or packages may be provided to a practitioner that are adapted for a particular use. For example, a package may be provided that includes a bolster device as described for use in the neck area, or devices for use in the hand or foot areas or in any of the areas described herein or shown in the attached drawings. It is a further aspect of such embodiments, that a bilaminate structure designed for the entire hand or any other area may be provided and the physician may cut out and use only the necessary parts of the structure. It is also contemplated that the devices may be provided in various sizes, such as small, medium and large in any type of device, or designed for individual fingers, or for a selected gender where appropriate.

Any and all such embodiments are within the content and spirit of the present disclosure. Also embodied herein are methods of use of the disclosed devices. For example, an aspect of the disclosure is a method of protecting a surgical wound or a skin graft by providing the appropriate bolster and applying it to the wounded or grafted area as described.

The following example is included to demonstrate a preferred embodiment of the invention. It should be appreciated by those of skill in the art that the techniques disclosed in the example which follows represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE

A 71 year old woman with a history of leukoplakia of the tongue and oral mucosa was treated 12 months previously with radiotherapy (50 Gy) for a T3N0M0 squamous cell carcinoma (SCC) of the left tonsilar fossa. She subsequently developed a second primary T1N0M0 SCC of the dorsal tongue and underwent partial (central) glossectomy and subtotal glossal mucosectomy. The defect was reconstructed with STSG (100 cm², 12/1000th inch thickness) which extended over the majority of the dorsal and ventral surface. The STSG was not meshed or fenestrated, normally rendering the graft more susceptible to hematoma, seroma or graft failure. The graft was inset followed by placement of a “tongue sandwich” bolster. The bolster was removed 5 days postoperatively at the bedside demonstrating over 95 percent survival of the graft. The patient was advanced to a regular diet over several days without difficulty and maintained intelligible speech. However, she continued to have limitation in tongue projection over the follow-up period of 9 months.

FIG. 1 shows an exploded perspective view of a bolster as described in the present example. This drawing depicts an embodiment in which two bolsters are attached to opposing sides of a graft. The patient's tongue 10 includes an area containing a split thickness skin graft 12. The graft gives a quilted appearance to the tongue. Upper 14 and lower 16 non-adherent gauze sections are applied directly over the skin grafted tongue.

The lower bolster 20 is cut to remove a central portion 13 to accommodate the root or base 11 of the tongue 10. Both components 18 and 20 may be cut to fit the particular patient's tongue and oral opening. In applying the devices, the upper 18 and lower 20 bolster components are placed on the respective gauze sections 14, 16 to form a “tongue sandwich.” The bolster components are then sutured in place using transglossal, silk sutures 30.

Upper 18 and lower 20 components are each bi-layered laminates. The semi-rigid outer layers 22A and 22B are made of a semi-rigid polyethylene foam composition. The inner layers 24A and 24B are made of a soft, compressive, sterile polyurethane foam. The layers are bonded together with an appropriate adhesive.

The tongue sandwich bolster provides a uniform compression over the entire graft surface, causing the graft to splay for maximum surface area. The inner foam layers 24A and 24B evenly distribute surface tension to the surface of the graft and conform to the irregular contour of the tongue. The semi-rigid, outer foam allows for the sandwich to be compressed, thereby immobilizing and splaying the tongue to its maximal surface area.

The initial bolster components 18 and 20 constructed for the present example were constructed by laminating sterile polyurethane foam (Reston Products®, 3M Health Care) to a semi-rigid, outer polyethylene foam insert from a sterile needle and sharps disposal unit (Pop-N-Count®, Kendall, L. T. P., Mansfield, Mass.). This approximately 1 cm thick foam block had an adhesive on one surface and a printed, numbered, grid on the other. Using a scalpel blade both surfaces are removed and the foam was thinned to create the specific rigidity and flexibility required. The polyurethane foam had adhesive on one surface allowing rapid and simple lamination. Separate upper and lower bilayer composite foam bolster components 18 and 20 were fabricated to the appropriate size and shape with scissors. The lower component 20 was fashioned in a “U” shape to surround the root of the tongue. The STSG was inset with 5/0 chronic sutures as well as multiple quilting sutures. A non-adherent gauze (N-Terface®, Winfield Laboratories, Inc., was applied directly over the skin grafted surface. The dorsal and ventral bolster components were secured with 3 transglossal, through-and-through 2/0 silk sutures, compressing and immobilizing the skin grafted tongue in a “sandwich” configuration.

The bolster components are easily constructed in approximately 5 minutes and fixation is achieved with 3 transglossal sutures. In this particular reported case an extensive tongue surface defect was created and the entire tongue, splayed by the disclosed bolster and method, could not be completely contained within the oral cavity. This was tolerated well by the patient and was easily removed. The ungrafted tip of the tongue and lips received periodic application of antibiotic ointment to prevent desiccation. Despite previous radiotherapy, the STSG had excellent take with no occurrence of hematoma or seroma when applied to a large, irregular graft surface involving the dorsal and ventral surfaces of the tongue. Contraction limited tongue projection; however, swallowing was unaffected and speech intelligibility was maintained.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined herein.

1. A bolster dressing to secure skin grafts or protect wounds of a patient, said bolster dressing comprising an outer semi-rigid layer connected with an inner conformable layer whereby said inner conformable layer is disposed between said outer semi-rigid layer and the body.

2. The bolster dressing of claim 1 wherein the outer semi-rigid layer is conformable to the contour of the body.

3. The bolster dressing of claim 1 wherein the outer semi-rigid layer is composed of one or more materials selected from the group consisting of semi-rigid polyethylene foam, semi-rigid polymer, plastic, wood, or metal.

4. The bolster dressing of claim 1 wherein the inner conformable material is composed of one or more materials selected from the group consisting of polyurethane foam, semi-open celled foam, silicone gel, cotton, wool, gauze impregnated with petroleum jelly, woven synthetic materials, unwoven synthetic materials, and a vacuum assisted closure dressing.

5. The bolster dressing of claim 1 wherein the inner conformable material is absorbent.

6. The bolster dressing of claim 1 wherein the inner conformable material is non-absorbent.

7. The bolster dressing of claim 1 further comprising a non-adherent layer disposed between said inner conformable layer and the body.

8. The bolster dressing of claim 1 further comprising a drug delivery layer for an active agent to be delivered to the body.

9. The bolster dressing of claim 8 wherein the drug delivery layer is disposed between the outer semi-rigid layer and the inner conformable layer.

10. The bolster dressing of claim 8 wherein the drug delivery layer is disposed between the inner conformable layer and the body.

11. The bolster dressing of claim 10 further comprising a non-adherent layer disposed between the drug delivery layer and the body.

12. The bolster dressing of claim 8 wherein the drug delivery layer and the inner conformable layer constitute one layer.

13. The bolster dressing of claim 8 wherein the drug delivery layer is biodegradable.

14. The bolster dressing of claim 8 wherein the drug delivery layer comprises an active agent contained by one or more materials chosen from the group consisting of solvent based polymers, water based polymers, microcapsules, microspheres, membranes, gels, hydrogels, liposomes, cellulose, glycosaminoglycans, carbohydrate polymers, and protein polymers.

15. The bolster dressing of claim 8 wherein the active agent is one or more agent chosen from the group consisting of drugs, hormones, antibiotics, growth factors, cytokines, chemotherapeutic agents, antiseptics, anesthetics, vitamins, minerals, cellular nutrients, co-factors, and antioxidants.

16. The bolster dressing of claim 8 wherein the active agent is released over a period of time.

17. The bolster dressing of claim 8 wherein the drug delivery layer is replenishable without removing the bolster dressing from the body.

18. The bolster dressing of claim 1 wherein the inner conformable layer is connected with the outer semi-rigid layer with one or more of adhesive, epoxy, or heat meld.

19. The bolster dressing of claim 1 wherein the bolster dressing is attached to the body using sutures, surgical staples, adhesive film, wrapping, tape, an elastic material, cast, splint, silicone adhesive, or a vacuum assisted closure device.

20. The bolster dressing of claim 1 further comprising a vacuum assisted closure dressing.

21. The bolster dressing of claim 20 wherein the vacuum assisted closure dressing is connected with a vacuum assisted closure device to provide negative pressure to the vacuum assisted closure dressing.

22. A bolster dressing of claim 1 preshaped to match a part of the body.

23. A bolster dressing of claim 22 wherein the part of the body is chosen from the group consisting of the face, an ear, a nose, a jaw, a cheek, a lip, an oral cavity, a neck, the back, the sacral region, an arm, an axilla, an elbow, a hand, a finger, a leg, a knee, a foot, a toe, a groin, a vagina, a penis, a buttock, and a perineum.

24. A bolster dressing of claim 22 wherein the part of the body is a tongue.

25. A bolster dressing of claim 22 wherein the part of the body is a nipple areola.

26. A sterile medical package comprising a bolster dressing wherein the package comprises:
(a) an inner conformable layer; and
(b) an outer semi-rigid layer configurable to match the contours of a body.

27. A sterile medical package of claim 26 wherein the inner conformable layer and the outer semi-rigid layer are separate and can be attached to one another by a user at the time of use.

28. A sterile medical package of claim 26 wherein the inner conformable layer and the outer semi-rigid layer are preshaped to match a body part chosen from the group consisting of face, ear, nose, jaw, cheek, lip, tongue, oral cavity, neck, back, sacral region, nipple areola, arm, axilla, elbow, hand, finger, leg, knee foot, toe, groin, vagina, penis, buttock or perineum.

29. A sterile medical package of claim 26 wherein the outer semi-rigid layer is a flat sheet to be cut and formed to fit a body by a user during use.

30. A sterile medical package of claim 26 wherein the inner conformable layer is a flat sheet to be cut and formed to fit a body by a user during use.

31. A sterile medical package of claim 26 further comprising a non-adherent layer wherein the non-adherent layer is a flat sheet to be cut and formed to fit a body by a user during use.

32. A sterile medical package of claim 26 further comprising a drug delivery layer wherein the drug delivery layer is a flat sheet to be cut and formed to fit a body by a user during use.

33. A bolster dressing to secure skin grafts or protect wounds of a patient comprising a semi-rigid polyethylene foam layer adhered to a compressive polyurethane foam layer and a non-adherent silicone film wherein the compressive polyurethane layer is disposed between the semi-rigid polyethylene layer and the non-adherent silicone film and the non-adherent silicone film contacts the body of the patient.

34. A method of securing a skin graft or protecting a wound of the body of a patient comprising:
(a) applying a layer of conformable material to the body;
(b) shaping a layer of semi-rigid material to the contour of the body;
(c) applying the layer of semi-rigid material so shaped over the layer of conformable material;
(d) attaching the layer of conformable material and the layer of semi-rigid material to the body to provide a substantially uniform compressive force to the skin graft or wound of the body.

35. The method of claim 34 further comprising the step of applying a layer of non-adherent material between the body and the layer of conformable material.

36. The method of claim 34 further comprising applying a drug delivery layer between the layer of semi-rigid material and the body.
